(19) United States
(12) Patent Application Publication
(10) Pub. No.: US 2024/0331149 A1
(43) Pub. Date: Oct. 3, 2024

(54) RECORDING MEDIUM, MEDICAL IMAGE DISPLAY APPARATUS, AND MEDICAL IMAGE DISPLAY METHOD

(71) Applicant: Konica Minolta, Inc., Tokyo (JP)

(72) Inventors: Hisashi Wada, Tyoko (JP); Hidekazu Takahashi, Osaka (JP); Ken Satou, Tokyo (JP)

(73) Assignee: Konica Minolta, Inc., Tokyo (JP)

(21) Appl. No.: 18/620,023

(22) Filed: Mar. 28, 2024

(30) Foreign Application Priority Data
Mar. 28, 2023 (JP) 2023-050743

(51) Int. Cl. G06T 7/00; G16H 15/00

(52) U.S. Cl. CPC G06T 7/0012; G16H 15/00; G06T 2207/30096

(57) ABSTRACT

A non-transitory computer readable recording medium stores instructions causing a computer to execute: extracting, from a medical image, an image region including a lesion candidate region obtained by processing the medical image; causing a display to display the image region; and identifying a confirmation result of the image region.

### CROSS-REFERENCE TO RELATED APPLICATIONS

The entire disclosure of Japanese Patent Application No. 2023-050743 filed on Mar. 28, 2023, including description, claims, drawings and abstract is incorporated herein by reference.

### BACKGROUND

### Technical Field

The present invention relates to a recording medium, a medical image display apparatus, and a medical image display method.

### Description of Related Art

Conventionally, it has been known that in medical image interpretation diagnosis, a secondary interpretation doctor confirms an interpretation diagnosis result by a primary interpretation doctor and makes definite diagnosis. In recent years, a diagnosis support (CAD: Computer Aided Detection/Diagnosis) system that causes a computer to analyze a medical image, and allows a doctor to diagnose a lesion candidate region has been put into practical use.

JP 2005-56296A describes a medical image report creation apparatus as described below. Specifically, first, a CAD detects an abnormal site, based on an image in an image management server. Next, the CAD only extracts a slice where the abnormal site is detected, attaches the slice as a reference image to a report, and transfers the report to the report creation apparatus.

However, according to a conventional interpretation diagnosis method, an interpretation diagnosis result by a primary interpretation doctor may sometimes be wrong. In this case, the interpretation diagnosis result by the primary interpretation doctor cannot be adopted as it is. Accordingly, confirmation work by a secondary interpretation doctor correcting and making addition to an interpretation report created by the primary interpretation doctor, and a diagnostic image annotated by the primary interpretation doctor, is required. The CAD system described in JP 2005-56296A has a low computer analysis accuracy, and a false positive may sometimes be included. Accordingly, also in this case, the interpretation doctor's confirmation work for a diagnosis result by AI is required. As in these cases, if the interpretation result by the primary interpretation doctor or the computer cannot be adopted as it is, there is a technical problem that confirmation work by a secondary interpretation doctor becomes complicated.

### SUMMARY

Accordingly, one or more embodiments of the present invention provide a recording medium, a medical image display apparatus, a medical image display system, and a medical image display method that improve functionalities of the apparatus/system, especially graphics techniques by displaying a result of a primary diagnosis to enable confirmation work for a secondary diagnosis, and thus provide technical improvements in medical image interpretation diagnosis technology.

According to an aspect of the present invention, a non-transitory computer readable recording medium stores:
instructions causing a computer to execute:
extracting, from a medical image, an image region including a lesion candidate region obtained by processing the medical image;
causing a display to display the image region; and
identifying a confirmation result of the image region.

According to an aspect of the present invention, a medical image display apparatus includes a hardware processor that:
extracts, from a medical image, an image region including a lesion candidate region obtained by processing the medical image;
causes a display to display the extracted image region; and
identifies a confirmation result of the image region.

According to an aspect of the present invention, a medical image display method causes a hardware processor to execute:
extracting, from a medical image, an image region including a lesion candidate region obtained by processing the medical image;
causing a display to display the image region; and
identifying a confirmation result of the image region.

### BRIEF DESCRIPTION OF THE DRAWINGS

The advantages and features provided by one or more embodiments of the invention will become more fully understood from the detailed description given hereinbelow and the appended drawings which are given by way of illustration only, and thus are not intended as a definition of the limits of the present invention, wherein:

FIG. 1 shows a schematic configuration example of a medical image display system according to a first embodiment;

FIG. 2 is a block diagram of a medical image management server according to the first embodiment;

FIG. 3 shows a configuration example of a data management table of the medical image management server according to the first embodiment;

FIG. 4 is a block diagram of an information processing apparatus according to the first embodiment;

FIG. 5 shows a configuration example of an interpretation screen displayed on a display of the information processing apparatus according to the first embodiment;

FIG. 6 is a flowchart showing an operation example of a medical image management server in a medical image analysis process according to the first embodiment;

FIG. 7 is a flowchart showing an operation example of the information processing apparatus in an interpretation support process according to the first embodiment;

FIG. 8A shows a display example of image regions including lesion candidate regions visually listed on the interpretation screen according to the first embodiment;

FIG. 8B shows an input method for a confirmation instruction using a menu according to the first embodiment;

FIG. 9 shows a display example of indications displayed on the image regions including the lesion candidate regions according to the first embodiment;

FIG. 10 shows a configuration example of an interpretation screen displayed on a display of an information processing apparatus according to a second embodiment;

FIG. 11 is a flowchart showing an example of operation of the information processing apparatus in an interpretation support process according to the second embodiment;

FIGS. 12A-12E show a display example of image regions including lesion candidate regions displayed in a sequentially switched manner on the interpretation screen according to the second embodiment; and

FIG. 13 shows a display example of image regions including lesion candidate regions visually listed on an interpretation screen according to a third embodiment.

### DETAILED DESCRIPTION

Hereinafter, embodiments of the present invention will be described with reference to the drawings. However, the scope of the invention is not limited to the disclosed embodiments.

### Configuration Example of Medical Image Display System 100

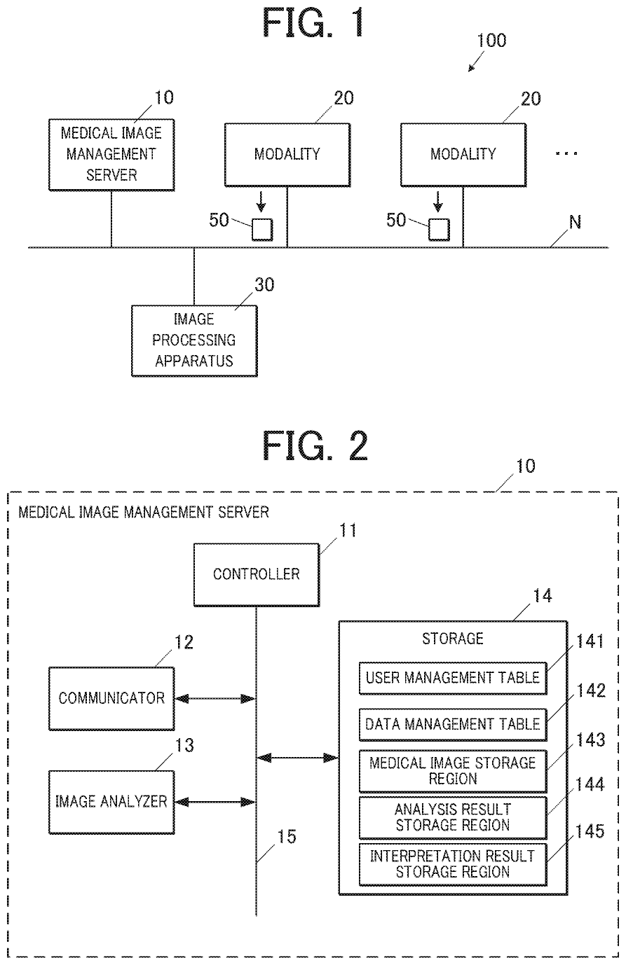

FIG. 1 shows a schematic configuration example of a medical image display system 100 according to the present embodiment.

As shown in FIG. 1, the medical image display system 100 includes: a medical image management server 10; modalities 20, 20, . . . , which are examples of imaging apparatuses; and an information processing apparatus 30, which is an example of a computer and a medical image display apparatus. The medical image management server 10, the modalities 20, and the information processing apparatus 30 are connected in a data-communicable manner to each other via a communication network N. Each apparatus included in the medical image display system 100 conforms to the Health Level Seven (HL7) or Digital Image and Communications in Medicine (DICOM) standards. Communication between the individual apparatuses is performed in conformity with HL7 or DICOM. Note that the number of information processing apparatuses 30 is not specifically limited. The communication network N is, for example, a local area network (LAN), a wide area network (WAN), the Internet, etc. The communication scheme may be a wired or wireless one as long as stable communication can be achieved.

The modality 20 takes a medical image 50 of a patient (subject), and generates image data of the taken medical image 50 of the patient. The modalities 20 may be, for example, of computed radiography (CR), digital radiography (DR), computed tomography (CT), magnetic resonance imaging (MRI), ultra sonography (US), nuclear medicine (NM), and endoscope(ES) for endoscopy. The modality 20 adds image attribute information pertaining to the taken medical image 50, to this taken medical image 50. The image attribute information includes the patient ID, patient name, birth date, gender, imaging time and date, image ID, modality, site, and direction. The modality 20 transmits the generated image data of the medical image 50, and the image attribute information added to the image data, to the medical image management server 10.

The medical image management server 10 stores and manages the image data of the medical image 50 generated by the modality 20, the image attribute information and the like. The medical image management server 10 may be, for example, a picture archiving and communication system (PACS) etc.

The information processing apparatus 30 is a computer apparatus, such as a personal computer (PC), or a tablet. The information processing apparatus 30 is used when a user, such as a radiographer, an interpretation doctor, or a clinician, interprets the medical image 50. The information processing apparatus 30 obtains the image data of the medical image 50 from the medical image management server 10, and displays the medical image 50, based on the obtained image data.

### Configuration Example of Medical Image Management Server 10

FIG. 2 is a block diagram of the medical image management server 10 according to the present embodiment. The medical image management server 10 includes a controller 11, a communication interface 12, an image analyzer 13, and a storage 14. The controller 11, and components such as the communication interface 12 are connected to each other by a bus 15.

The controller 11 includes a processor, such as a central processing unit (CPU), and a memory, such as a random access memory (RAM). The CPU of the controller 11 reads various instructions such as processing programs stored in the storage 14, and achieves various functions related to the medical image management server 10 through cooperation with the instructions. The CPU of the controller 11 may include a single processor, or include a plurality of processors, which may operate in parallel or independently for each application.

The communication interface 12 includes a network interface. The communication interface 12 transmits and receives various types of data to and from the modalities 20 and the information processing apparatus 30, which are connected to each other via the communication network N. For example, the communication interface 12 receives the image data of the medical image 50 of the patient taken by the modality 20. When an obtainment request for the medical image 50 is transmitted from the information processing apparatus 30, the communication interface 12 transmits, to the information processing apparatus 30, the medical image 50 in response to the obtainment request, and an analysis result on a lesion candidate on the medical image 50.

The image analyzer 13 executes computer processing to the medical image 50 taken by the modality 20, and detects a lesion candidate region from the medical image 50. The lesion candidate region is an image region including, for example, a nodular shadow that is a sign suspicious of lung cancer, an infiltrative shadow that is a sign of an infection, such as pneumonia or tuberculosis, etc. The image analyzer 13 generates analysis result data that includes the detected lesion candidate region. For example, AI analysis (computer aided diagnosis (CAD)) utilizing artificial intelligence (AI) that detects a lesion candidate from the medical image 50 and performs image diagnosis and image analysis can be used as the computer processing. The analysis result data includes, for example, DICOM grayscale softcopy presentation state (GSPS) data, overlay data, etc. The analysis result data includes an analysis result ID for identifying the analysis result, and various types of information about the lesion candidate region. The various types of information on the lesion candidate region include, for example, the position, lesion type, and content of added information (annotation information). The image analyzer 13 is achieved by software processing through cooperation between the instructions stored in the storage 14 and the CPU of the controller 11. Note that a part of or the entire image analyzer 13 may be implemented in the controller 11, or may be achieved by an external apparatus or a cloud on the network, for example. The image analyzer 13 may be provided with a learning function of learning a technique of interpretation diagnosis of doctors by machine learning, such as deep learning, thus improving the capability of analyzing the lesion on the medical image 50. The learning function may be achieved by, for example, an external apparatus or a cloud on the network different from the information processing apparatus 30.

The storage 14 includes at least one of a semiconductor memory, a hard disk drive (HDD), an optical disk storing device and the like. The storage 14 stores various instructions such as processing programs, and parameters and files required to execute the instructions. The storage 14 includes a user management table 141, a data management table 142, a medical image storage region 143, an analysis result storage region 144, and an interpretation result storage region 145.

The user management table 141 is a table for managing users (healthcare workers, such as doctors) using the medical image display system 100. The user management table 141 stores a user ID, password, name, affiliation, email address, phone number and the like in association with each other, for each user.

The user ID is identification information on the user. The password is used for authentication when the user accesses the medical image management server 10 from the information processing apparatus 30. The name is the name of the user. The affiliation is information on a medical facility, a clinical department and the like to which the user belongs. The email address is an email address of the user. The phone number is a phone number of the user.

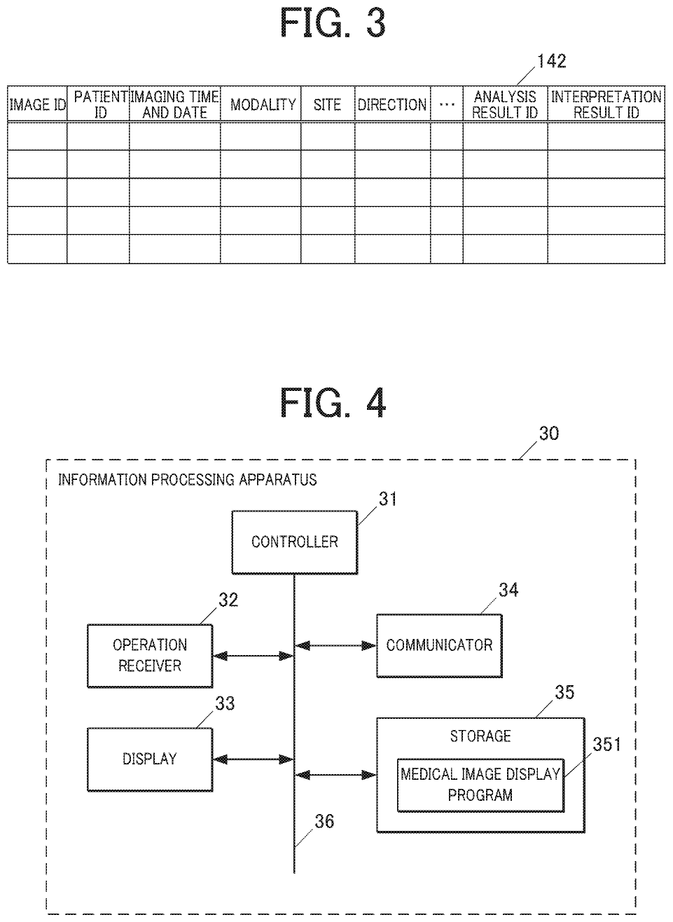

The data management table 142 is a table for managing data in the medical image management server 10. FIG. 3 shows an example of the configuration of the data management table 142 according to the present embodiment. As shown in FIG. 3, the data management table 142 stores an image ID, patient ID, imaging time and date, modality, site, direction, analysis result ID, interpretation result ID and the like in association with each other, for each medical image 50 stored in the medical image storage region 143.

The image ID is identification information on the medical image 50. The patient ID is identification information on the patient as the imaging target of the medical image 50. The imaging time and date is the time and date of taking the medical image 50. The modality is a modality that has taken the medical image 50. The site is a site that is the imaging target of the medical image 50. The direction is the imaging direction of the medical image 50. The analysis result ID is identification information on an analysis result obtained by the image analyzer 13 analyzing the medical image 50. The interpretation result ID is identification information for identifying an interpretation result for the medical image 50 by the user.

The medical image storage region 143 stores image data of the medical image 50 taken by the modality 20. The analysis result storage region 144 stores the analysis result data that includes the lesion candidate region obtained by applying computer processing to the medical image 50. The interpretation result storage region 145 stores data of the interpretation result of the medical image 50 by the user. The interpretation result includes, for example, confirmation result information indicating that it has been confirmed (approved) whether the analysis result determined to be the lesion candidate region by the computer processing is right (secondary positive) or not right (secondary false positive). The confirmation result information includes, for example, the position of the lesion candidate region, lesion type, and added information (e.g., annotation information).

### Configuration Example of Information Processing Apparatus 30

FIG. 4 is a block diagram of the information processing apparatus 30 according to the present embodiment.

As shown in FIG. 4, the information processing apparatus 30 includes a controller 31 (i.e., hardware processor), an operation receiver 32, a display 33, a communication interface 34, and a storage 35. The controller 31, and components, such as the operation receiver 32, are connected to each other via a bus 36.

The controller 31 includes a processor, such as a CPU, and a memory, such as a RAM. The CPU of the controller 31 reads various instructions such as processing programs that include a medical image display program 351 stored in the storage 35, and achieves various functions related to the information processing apparatus 30 through cooperation with the instructions. The CPU of the controller 31 may include a single processor, or include a plurality of processors, which may operate in parallel or independently for each application.

The operation receiver 32 includes, for example, a keyboard that includes cursor keys, alphanumeric input keys, and various functional keys, and a pointing device, such as a mouse. The operation receiver 32 accepts various instructions input by the user through an operation on the keyboard or the like, and outputs operation signals in response to the accepted instructions to the controller 31. If the operation receiver 32 has a function of a touch panel, the operation receiver 32 accepts an instruction in accordance with the position of a touch operation by a finger or the like of the user.

The display 33 includes a display that is a liquid crystal display or an organic electroluminescence (EL) display. The display 33 displays various screens including an interpretation screen 331 used when the user performs interpretation diagnosis, based on display control by the controller 31. As described above, the display 33 may be a combination with a touch panel that is the operation receiver 32.

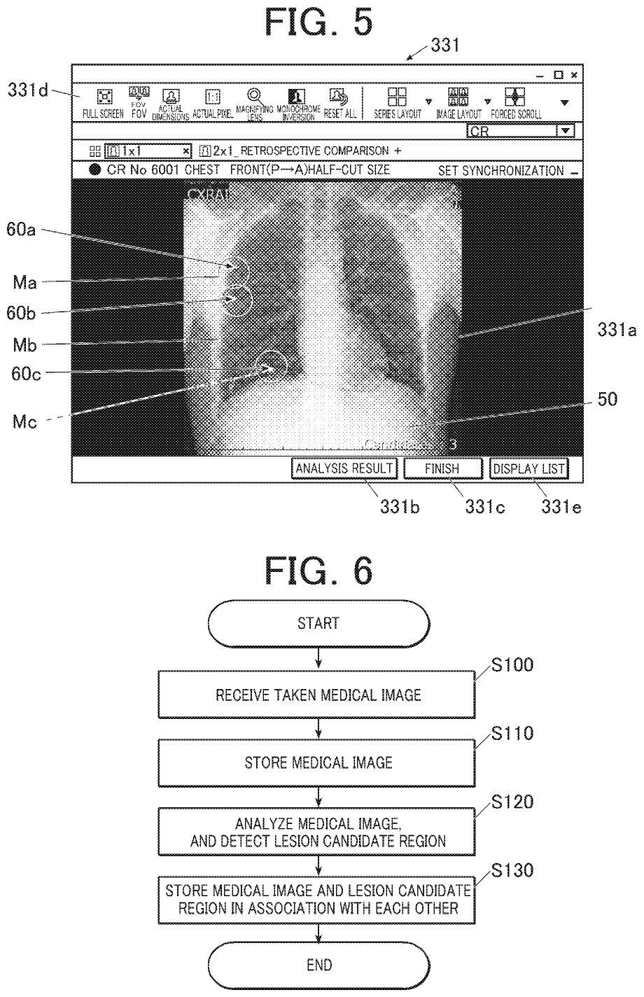

FIG. 5 shows an example of the configuration of the interpretation screen 331 displayed on the display 33 according to the first embodiment.

The interpretation screen 331 is a screen for allowing the user to visually perform interpretation diagnosis, and input an interpretation diagnosis result. The interpretation screen 331 is provided with a medical image display region 331a where a medical image 50 of a specific patient obtained from the medical image management server 10 is displayed. An upper part of the interpretation screen 331 is provided with a tool display region 331d that includes various types of buttons for editing a display method and the like for the medical image 50. A lower part of interpretation screen 331 is provided with an analysis result button 331b, a finish button 331c, and an image list display button 331c. The analysis result button 331b is a button for displaying the lesion candidate region detected through AI analysis by the computer, on the medical image 50. The finish button 331c is a button for issuing an instruction for finishing the interpretation diagnosis. The image list display button 331e is a button for visually listing an image region that includes lesion candidate regions detected through AI analysis by the computer.

As shown in FIG. 5, the medical image 50 is displayed in the medical image display region 331a on the interpretation screen 331. If there is a lesion candidate detected through AI analysis by the computer, a lesion candidate region 60 is displayed on the medical image 50. In the present embodiment, as an example, two locations of a first lesion candidate region 60a and a second lesion candidate region 60b are displayed. The first lesion candidate region 60a and the like indicate the position, size etc. of the lesion candidate. For example, the first lesion candidate region 60a is encircled by a white circular mark Ma that indicates the position of a lesion candidate. The second lesion candidate region 60b is encircled by a white circular mark Mb that indicates the position of a lesion candidate. Accordingly, the user can easily identify the lesion candidate regions 60 detected through AI analysis by the computer on the medical image 50. The marks for identifying the positions of the lesion candidates are not specifically limited to circular marks as long as the marks can identify the lesion candidates and do not disturb interpretation. Hereinafter, the first lesion candidate region 60a, the second lesion candidate region 60b and the like may be collectively referred to as the lesion candidate region 60.

Referring back to FIG. 4, the communication interface 34 includes a network interface. The communication interface 34 transmits and receives various types of data to and from the medical image management server 10 and the modalities 20, which are connected to each other via the communication network N.

The storage 35 includes at least one of a semiconductor memory, a hard disk drive (HDD), an optical disk recording device and the like. The storage 35 stores various instructions such as processing programs, and parameters and files required to execute the instructions. The storage 35 stores the medical image display program 351. The medical image display program 351 is a program for allowing the user to efficiently perform interpretation and determination of an interpretation result of the medical image 50 analyzed by the computer.

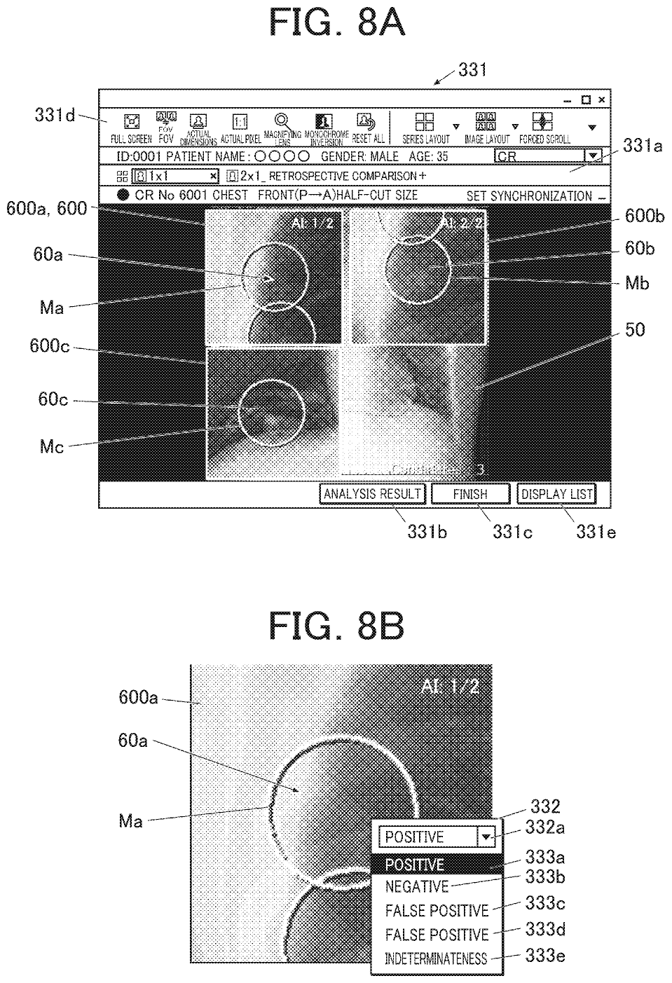

In the information processing apparatus 30 described above, the controller 31 achieves the following functions by executing the medical image display program 351. The controller 31 functions as an extractor, and executes a first extracting function and an extracting step that extract, from the medical image 50, image regions 600 (see FIG. 8A) including lesion candidate regions 60 obtained by computer processing to the medical image 50. Here, each image region 600 is a partial region extracted from the medical image 50. The controller 31 functions as a display controller, and executes a display function and a display step that cause the display 33 to visually list the image regions 600 extracted by the first extracting function. The controller 31 functions as an identifier, and executes an identification function and an identification step that identify a confirmation result of the image region 600 extracted by the first extracting function. The confirmation result includes an interpretation determination result of the lesion candidate region in the image region 600 extracted by the first extracting function.

### Operation Example of Medical Image Management Server 10

FIG. 6 is a flowchart showing an operation example of the medical image management server 10 in a medical image analysis process according to the first embodiment. This process is achieved by software processing through cooperation between the CPU of the controller 11 and the instructions stored in the storage 14.

The communication interface 12 of the medical image management server 10 receives, via the network N, the medical image 50 of the patient taken by the modality 20 (Step S100).

The controller 11 stores the medical image 50 of the patient received from the modality 20, in the medical image storage region 143 in the storage 14 (see FIG. 2) (Step S110). The controller 11 stores, in the data management table 142 in the storage 14 (see FIG. 2), the image ID, patient ID, imaging time and date, modality, site, direction and the like included in the image attribute information on the medical image 50 received from the modality 20.

The image analyzer 13 analyzes the medical image 50 received from the modality 20 through artificial intelligence (AI), thereby detecting the lesion candidate region 60 suspicious of lung cancer, pneumonia or the like from the medical image 50 (Step S120).

The controller 11 associates analysis result data including the lesion candidate region 60 analyzed by the image analyzer 13, with the medical image 50 that is the original image as an analysis target, and stores them in the storage 14 (Step S130). For example, the controller 11 stores the analysis result data in the analysis result storage region 144 (see FIG. 2).

### Operation Example of Medical Image Display System 100

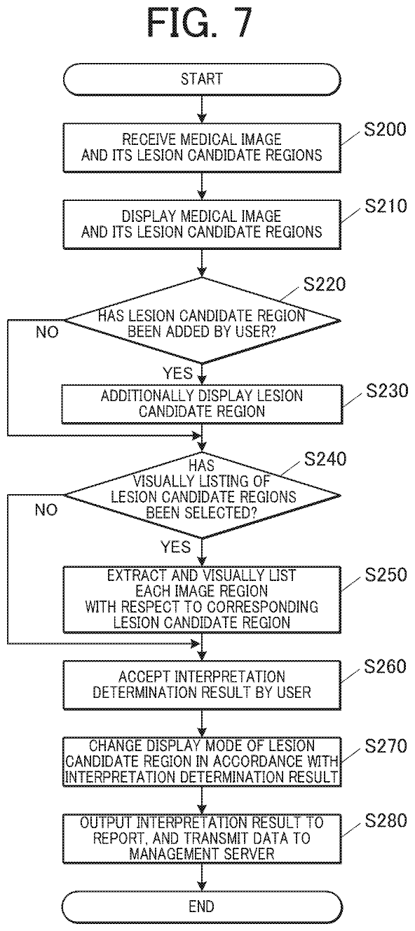

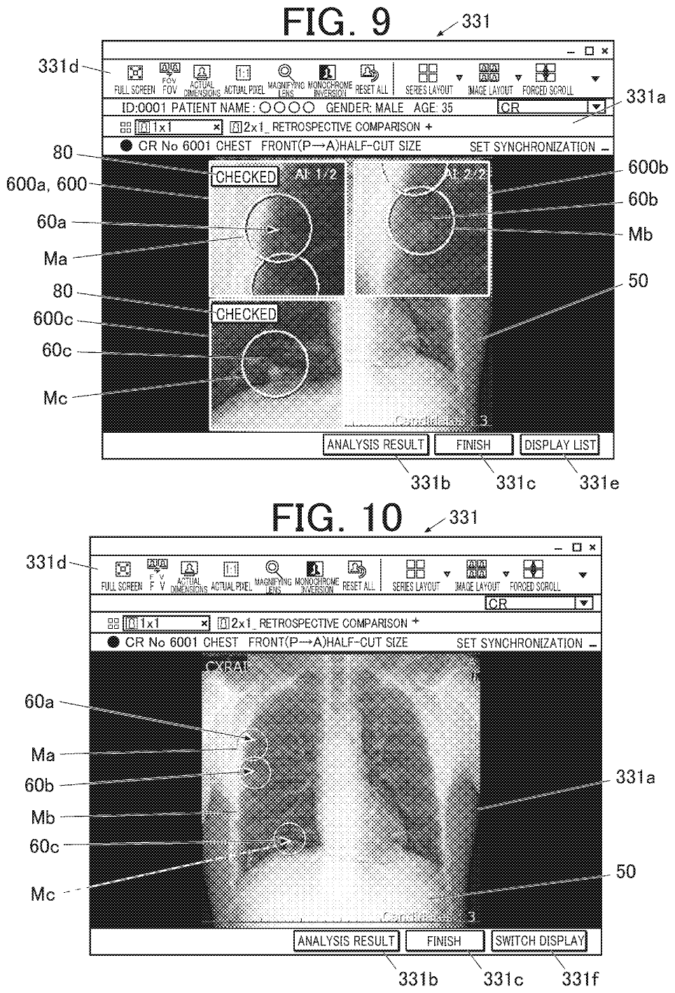

FIG. 7 is a flowchart showing an example of operation of the information processing apparatus 30 in an interpretation support process according to the first embodiment. FIG. 8A shows a display example of the image region 600 including the lesion candidate regions 60 visually listed on the interpretation screen 331 according to the first embodiment. FIG. 8B shows an example of an input method for a confirmation instruction using a menu 332. FIG. 9 shows a display example of indications (confirmation icon/box) 80 displayed on the image regions 600 including the lesion candidate regions 60. This process is achieved by software processing through cooperation between the CPU of the controller 31 and the medical image display program 351 stored in the storage 35. Note that a case where the plurality of lesion candidate regions 60 are included in the medical image 50 is hereinafter described.

As shown in FIG. 7, the controller 31 receives the medical image 50, and the analysis result data including the lesion candidate regions 60 associated with the medical image 50, from the medical image management server 10 (Step S200). Specifically, the controller 31 transmits an obtainment request for the medical image 50 including the image ID designated by the user, to the medical image management server 10. The controller 11 of the medical image management server 10 reads the medical image associated with the image ID included in the obtainment request, from the medical image storage region 143, and transmits the read medical image 50 to the information processing apparatus 30. The controller 11 refers to the data management table 142 in the storage 14, and obtains the analysis result ID from a record associated with the image ID of the medical image 50. The controller 11 reads the analysis result data associated with the identified analysis result ID from the analysis result storage region 144, and transmits the data to the information processing apparatus 30.

The controller 31 displays the medical image 50 obtained from the medical image management server 10, and the lesion candidate regions 60 included in the analysis result data, on the interpretation screen 331 of the display 33 (Step S210). Specifically, as shown in FIG. 5, the controller 31 displays the medical image 50, and the first lesion candidate region 60a and the second lesion candidate region 60b detected through AI analysis by the computer, on the interpretation screen 331.

The controller 31 determines whether an additional instruction for a new lesion candidate region 60 by the user has been accepted or not on the medical image 50 displayed on the interpretation screen 331 (Step S220). Specifically, if the user finds a new lesion candidate other than the first lesion candidate region 60a and the like detected through AI analysis by the computer on the medical image 50 displayed on the interpretation screen 331, the user inputs the additional instruction for the new lesion candidate. For example, the additional instruction may be input by positioning the cursor through a mouse operation at the position of a newly selected lesion candidate on the medical image 50, and left-clicking. The additional instruction may be input by positioning the cursor at the position of the newly selected lesion candidate on the medical image 50, right-clicking, and selecting an item of a new positive from a menu. In a case where the operation receiver 32 includes a touch panel, the additional instruction may be input by performing a direct touch operation at the position of the newly selected lesion candidate on the medical image 50.

If the controller 31 determines that the additional instruction for a new lesion candidate region 60 by the user has been accepted (Step S220: YES), the processing proceeds to Step S230. The controller 31 displays a new lesion candidate region 60 on the medical image 50 displayed on the interpretation screen 331 in an overlapping manner (Step S230). Specifically, as shown in FIG. 5, the controller 31 displays a new third lesion candidate region 60c on the medical image 50 displayed on the interpretation screen 331. The third lesion candidate region 60c is encircled by a mark Mc indicated by a white circular broken line. Note that the mark Mc may be of a solid line as with the mark Ma or the like. To distinguish from the first lesion candidate region 60a and the like by the computer processing, the mark Mc may have a different line thickness, or a different color.

On the other hand, if the controller 31 determines that designation of the lesion candidate region 60 by the user has not been newly accepted (Step S220: NO), the processing proceeds to Step S240. In other words, this is a case where in the interpretation diagnosis by the user, any new lesion candidate other than the first lesion candidate region 60a and the like detected through AI analysis by the computer has not been found.

The controller 31 determines whether a display instruction for visually listing the lesion candidate regions 60 has been accepted by the user or not (Step S240). If the controller 31 determines that the display instruction for visually listing has been accepted (Step S240: YES), the processing proceeds to Step S250. For example, the controller 31 determines based on whether the display instruction in accordance with a selected operation for the image list display button 331c on the interpretation screen 331 shown in FIG. 5. has been accepted or not.

The controller 31 extracts the lesion candidate regions 60 from the medical image 50 displayed on the interpretation screen 331, and visually lists the image regions 600 including the extracted lesion candidate regions 60, on the interpretation screen 331 (Step S250). Specifically, the controller 31 executes the first extracting function of extracting a first image region 600a including the first lesion candidate region 60a, and a second image region 600b including the second lesion candidate region 60b, from the medical image 50. The controller 31 executes a second extracting function of extracting a third image region 600c including the third lesion candidate region 60c, from the medical image 50. Note that hereinafter the first image region 600a, the second image region 600b, and the third image region 600c may be collectively referred to as the image region 600.

FIG. 8A shows a display example of the image regions 600 including the lesion candidate regions 60 visually listed on the interpretation screen 331 according to the first embodiment.

As shown in FIG. 8A, the controller 31 executes the display function of visually listing three locations of the extracted first image region 600a, second image region 600b, and third image region 600c, for example, in two rows and two columns, on the interpretation screen 331. Note that the method of arranging the image region 600 is not limited to the two rows and two columns. For example, any arrangement, such as of three rows and three columns, can be adopted. In the present embodiment, the extraction shape and extraction size of the image region 600 are those of an image region that has been cut out into a rectangular shape and includes the periphery of the lesion candidate region 60. However, there is no limitation to this.

The controller 31 accepts the confirmation instruction by the user in accordance with the interpretation determination result, for the lesion candidate regions 60 visually listed on the interpretation screen 331 (Step S260). The interpretation determination result includes the result (confirmation result) when the user verified whether the analysis result of the computer about the lesion candidate region 60 determined to be positive through AI analysis of the computer has an error. The interpretation determination result may be a result when the user verifies the lesion candidate region 60 determined to be positive by himself/herself. Specifically, the user performs interpretation diagnosis for the lesion candidate regions 60 visually listed on the interpretation screen 331. If the user's own interpretation determination result is the same as the AI analysis result by the computer, the user inputs a positive confirmation instruction indicating that it has been confirmed (approved) that the first lesion candidate region 60a is positive. If the user's own interpretation determination result is different from the AI analysis result by the computer, the user inputs a false positive confirmation instruction indicating that it has been confirmed that the second lesion candidate region 60b is a false positive. If the user adds a new lesion candidate having not been found through AI analysis by the computer, the user inputs a positive confirmation instruction indicating that it has been confirmed that the third lesion candidate region 60c is a new positive.

The confirmation instruction may be input by one operation (one-touch operation). For example, the positive confirmation instruction may be input by positioning the cursor in the lesion candidate region 60 through a mouse operation, and left-clicking. As for the input of the false positive confirmation instruction, in a case where the lesion candidate region 60 is not selected, i.e., no operation is made, the input of the false positive confirmation instruction may be assumed to be made. As for the input of the false positive confirmation instruction, in a case where the number of left clicking is different from that of the positive confirmation instruction input, the input of the false positive confirmation instruction may be assumed to be made.

Another input method for the confirmation instruction may use the menu 332. FIG. 8B shows an input method for a confirmation instruction using the menu 332 according to the first embodiment. As shown in FIG. 8B, when the cursor is positioned in the image region 600 (first image region 600a) for the interpretation determination result, and a right-clicking operation is made, the controller 31 displays the menu 332 on the image region 600. When an arrow button 332a of the menu 332 is selected, the controller 31 expands and displays the menu 332. The menu 332 is provided with a positive button 333a, a negative button 333b, a false positive button 333c, a false negative button 333d, and a pending button 333e, for input of interpretation determination results. When any of buttons, such as the positive button 333a, is selected from the menu 332 by the user, the controller 31 accepts a confirmation instruction in accordance with the selected button.

In the present embodiment, the controller 31 accepts a positive confirmation instruction for the first image region 600a. The controller 31 accepts a false positive confirmation instruction for the second image region 600b. The controller 31 accepts a new positive confirmation instruction for the third image region 600c. For example, not only the positive, and false positive confirmation instructions but also at least one of negative, false negative and pending confirmation instructions can be input as the interpretation determination result.

The controller 31 changes the display mode of the lesion candidate regions 60 visually listed on the interpretation screen 331, in accordance with the confirmation result input by the user (Step S270). In other words, the controller 31 executes the identification function of identifying the confirmation result that is the interpretation determination result for the lesion candidate region 60 input by the user. Subsequently, the controller 31 executes the display function of displaying the indication 80 for confirming a state of the identified lesion candidate region 60.

FIG. 9 shows a display example of the indication 80 displayed on the image region 600 including the lesion candidate region 60 according to the first embodiment.

For example, as shown in FIG. 9, upon acceptance of the positive confirmation instruction for the first lesion candidate region 60a, the controller 31 displays the indication 80 with “CHECKED” on the first image region 600a. The controller 31 associates the first lesion candidate region 60a, and the indication 80 with “CHECKED”, with each other on one-to-one basis, and displays them on the medical image 50 in an overlapping manner. Upon acceptance of the false positive confirmation instruction for the second lesion candidate region 60b, the controller 31 does not change the display mode of the second lesion candidate region 60b, and makes the indication 80 hidden. Upon acceptance of the positive confirmation instruction for the third lesion candidate region 60c, the controller 31 displays the indication 80 with “CHECKED” on the third image region 600c. The third lesion candidate region 60c and the indication 80 with “CHECKED” are associated with each other on one-to-one basis, and displayed on the medical image 50 in the overlapping manner. If the user finds a new third lesion candidate region 60c, the indication 80 may be displayed with “CHECKED (NEW)”, for example, for distinguishing from the positive determination by the computer processing. Note that in the present embodiment, the example where “CHECKED” is used as an example of the indication 80 is described. However, there is no limitation to this. For example, the indication 80 may be characters other than “CHECKED”, a symbol, a diagram or the like, or a combination thereof.

On the other hand, in Step S240, if the controller 31 determines that the display instruction for visually listing has not been accepted (Step S240: NO), the processing proceeds to Step S260. For example, this is a case where the user does not use a list display function for the lesion candidate regions 60, and performs interpretation diagnosis for the lesion candidate region 60 on the medical image 50 displayed on the interpretation screen 331 shown in FIG. 5. If there is a lesion candidate region 60 detected through AI analysis by the computer on the medical image 50, the user determines whether the positive determination for the lesion candidate region 60 is right or wrong. The user determines whether there is a new lesion candidate region 60 on the medical image 50 or not. A method similar to that in the visually listing case described above may be adopted as the input method for the interpretation determination result.

If the interpretation diagnosis of the medical image 50 by the user is finished, the controller 31 executes an output function of outputting the interpretation determination result of the image region to the interpretation report, and transmits data indicating the interpretation report and the interpretation determination result to the medical image management server 10 (Step S280). The interpretation report is, for example, what indicates presence or absence of abnormality from the medical image 50 by an interpretation doctor in the department of radiology in response to an examination request from a clinical department, and briefs the state and disorder of the patient. The interpretation report is used when an examining doctor feeds back the diagnosis result to the patient.

The controller 31 identifies the image region 600 where at least one of positive, false negative, new positive, pending and the like is selected as the interpretation determination result by the user, through the identification function. The controller 31 extracts the identified image region 600 by a third extracting function, and pastes the region as a reference image to the interpretation report. The interpretation report displays so as to allow the next examining doctor to recognize, for example, a positive (secondary interpretation confirmed), a false positive (secondary interpretation confirmed), or a new positive (added by a secondary interpretation doctor) in each lesion candidate region 60. For example, the controller 31 may extract only the positive and newly positive image regions 600, and paste the regions to the interpretation report. The determination of whether it is the positive image region 600 or the like may be performed in a display mode of the image region 600, for example, based on whether the indication 80 with “CHECKED” is displayed in the image region 600 or not.

Note that in the embodiments described above, as the interpretation determination result by the user for the lesion candidate regions 60 detected by the computer processing, the indications 80 with “CHECKED” are displayed on the image regions 600. However, there is no limitation to this. For example, the controller 31 may change the display state of at least one of the concentration, the display position, and the size of the image region 600 as an example of the indication, in accordance with the interpretation determination result by the user. At least one of the concentration, the display position, and the size of the image region 600, and the indication 80 with “CHECKED” described above may be combined.

Specifically, the controller 31 may display the first image region 600a determined to be positive by the user more brightly or darkly than the second image region 600b determined to be false positive by the user.

The controller 31 may arrange the first image region 600a determined to be positive in the interpretation determination result by the user, for example, on the left side, and arrange the second image region 600b determined to be false positive in the interpretation determination result by the user t, for example, on the right side.

The controller 31 may display the first image region 600a determined to be positive in the interpretation determination result by the user larger or smaller than the second image region 600b determined to be false positive in the interpretation determination result by the user.

In the case of visually listing a plurality of lesion candidate regions 60 on the interpretation screen 331, the controller 31 may change the display order, based on at least one of pieces of information on the lesion accuracy and the size of a lesion of each of the lesion candidate regions 60. For example, if the lesion accuracy is high in the order of the first lesion candidate region 60a, the third lesion candidate region 60c, and the second lesion candidate region 60b, the first lesion candidate region 60a, the third lesion candidate region 60c, and the second lesion candidate region 60b are displayed in this order from the left of the displayed list. The size of the lesion is similar to that in the case of the lesion accuracy of the lesion. In consideration of both the lesion accuracy and size of the lesion, the display order of the lesion candidate regions 60 may be changed.

As described above, according to the first embodiment, the functionalities of the apparatus/system, especially graphics techniques are improved by displaying the result of the primary diagnosis to enable confirmation work for the secondary diagnosis, and thus provide technical improvements in medical image interpretation diagnosis technology. Specifically, the user can perform interpretation while visually identifying the image regions 600 including the lesion candidate regions 60 visually listed on the interpretation screen 331. In other words, the list image where only the image regions 600 including the lesion candidate regions 60 are extracted from the medical image 50 is verified, thus allowing secondary interpretation. Accordingly, improvement in the user's interpretation working efficiency can be facilitated.

According to the first embodiment, in a case of interpretation and verification for the visually listed lesion candidate regions 60, the interpretation determination result of the interpretation can be input by a left-clicking operation or the like in the image region 600. In this case, the identification function of the controller 31 automatically identifies the interpretation determination result. Accordingly, the interpretation determination result in the interpretation can be easily input and improvement in the user's interpretation working efficiency can be facilitated.

According to the first embodiment, in response to input of the interpretation determination result by the user, an indication (indication 80) indicating that the user has confirmed the analysis result by the computer is displayed in the image region 600. Consequently, it is possible to easily identify whether the user has confirmed presence or absence of a positive in the lesion candidate region 60 or not.

### Second Embodiment

According to a second embodiment, the lesion candidate regions 60 detected from the medical image 50 can be displayed in a sequentially switched manner for each image. Note that the components and operations identical to those of the medical image display system 100 and the like according to the first embodiment described above are assigned the identical symbols, and detailed description thereof is omitted.

FIG. 10 shows a configuration example of the interpretation screen 331 displayed on the display 33 according to the second embodiment.

As shown in FIG. 10, instead of the image list display button 331e used in the first embodiment, a switch display button 331f is displayed on the interpretation screen 331. The switch display button 331f is a button for displaying the image regions including the lesion candidate regions detected through AI analysis by the computer in a manner of being sequentially switched one by one. The other components are similar to the components of the interpretation screen 331 in FIG. 5 described above.

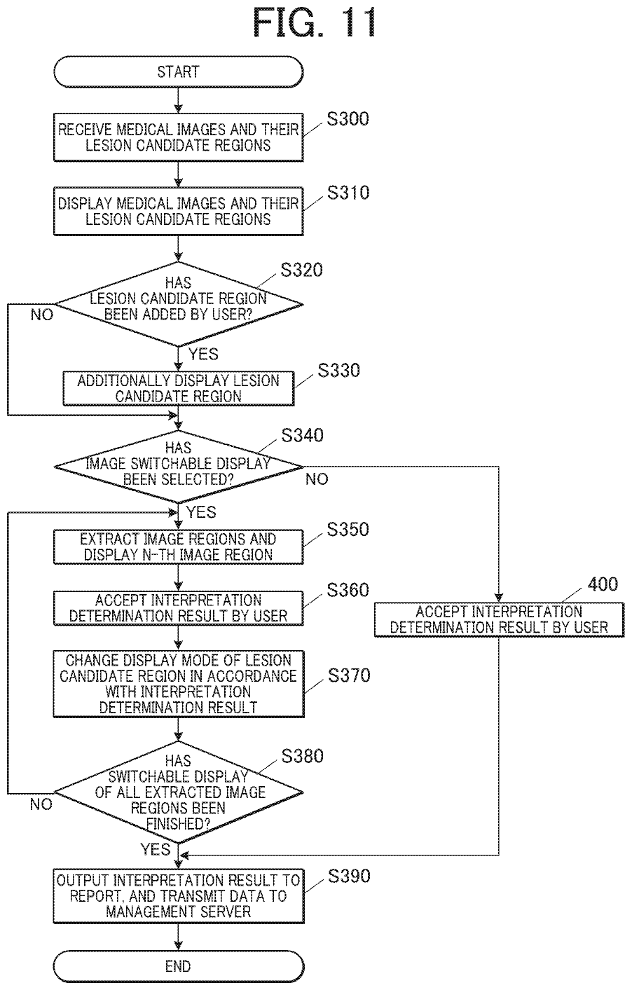

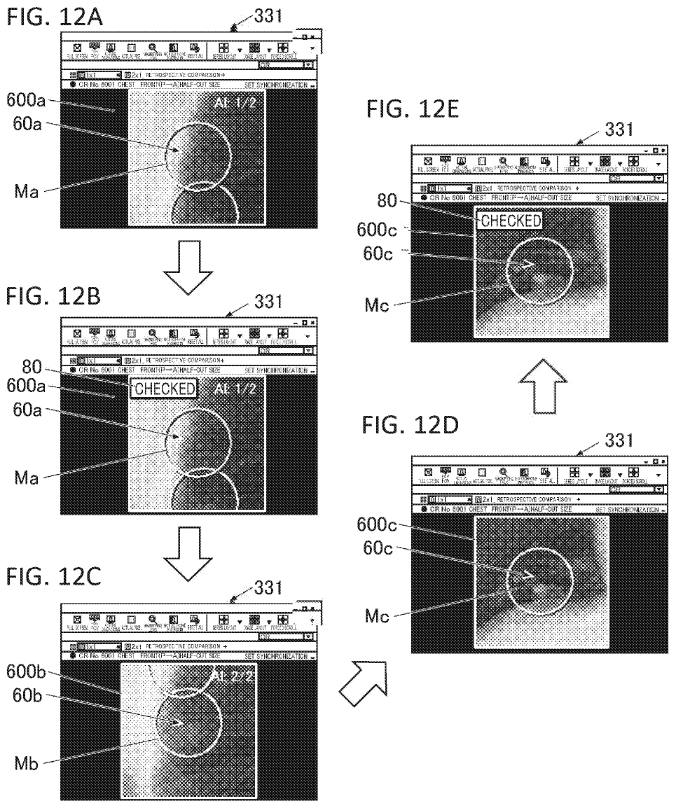

FIG. 11 is a flowchart showing an example of operation of the information processing apparatus 30 in an interpretation support process according to the second embodiment. FIGS. 12A-12E show a display example of the lesion candidate regions 60 displayed in a sequentially switched manner on the interpretation screen 331 according to the second embodiment. This process is achieved by software processing through cooperation between the CPU of the controller 31 and the instructions stored in the storage 35. Hereinafter, a case where lesion candidate regions 60 are included in medical image 50 is described. The types and number of lesion candidate regions 60 are similar to those in the first embodiment.

As shown in FIG. 11, the controller 31 receives the medical image 50, and the analysis result data including the lesion candidate regions 60 on the medical image 50, from the medical image management server 10 (Step S300).

The controller 31 displays the medical image 50 obtained from the medical image management server 10, and the lesion candidate regions 60 included in the analysis result data of the medical image 50, on the interpretation screen 331 of the display 33 (Step S310).

The controller 31 determines whether an additional instruction for a new lesion candidate region 60 by the user has been accepted or not on the medical image 50 displayed on the interpretation screen 331 (Step S320). If the controller 31 determines that the additional instruction for a new lesion candidate region 60 by the user has been accepted (Step S320: YES), the processing proceeds to Step S330. Upon acceptance of the user's additional instruction for a new lesion candidate region 60 by the user, the controller 31 displays the new lesion candidate region 60 on the medical image 50 displayed on the interpretation screen 331, in an overlapping manner, as shown in FIG. 5 (Step S330).

On the other hand, if the controller 31 determines that the additional instruction for the new lesion candidate region 60 by the user has not been accepted (Step S320: NO), the processing proceeds to Step S340.

The controller 31 determines whether a display instruction by the user for displaying the lesion candidate regions 60 in a sequentially switched manner has been accepted or not (Step S340). If the controller 31 determines that the display instruction for displaying in the sequentially switched manner has been accepted (Step S340: YES), the processing proceeds to Step S350.

The controller 31 extracts the image regions 600 including the lesion candidate regions 60 from the medical image 50 displayed on the interpretation screen 331, and displays the N-th image region among the extracted image regions 600 (Step S350). The variable N is a value corresponding to the number of extracted image regions 600, and for example, N=1 is set as an initial value. In the case where a plurality of lesion candidate regions 60 are present, numbers are sequentially assigned to the respective lesion candidate regions 60. In the present embodiment, No. 1 is assigned to the first image region 600a at the top on the medical image 50. No. 2 is assigned to the second image region 600b at the second from the top on the medical image 50. No. 3 is assigned to the third image region 600c at the third from the top on the medical image 50. As shown in FIG. 12A, since N=1 at the initial stage, the controller 31 displays the first image region 600a of No. 1 on the interpretation screen 331.

The controller 31 accepts the confirmation instruction by the user in accordance with the interpretation determination result, for the lesion candidate regions 60 displayed on the interpretation screen 331 in a switchable manner (Step S360). In the present embodiment, for example, a positive confirmation instruction for the first image region 600a is accepted.

The controller 31 changes the display mode of the lesion candidate regions 60 displayed on the interpretation screen 331, in accordance with the interpretation determination result input by the user (Step S370). For example, upon acceptance of the positive confirmation instruction for the first lesion candidate region 60a, the controller 31 displays the indication 80 with “CHECKED” on the first lesion candidate region 60a as shown in FIG. 12B, thus changing the display mode. When input of the interpretation determination result is finished, the user performs an operation, such as of pressing a button (not shown) for transition to the next image region, for example.

The controller 31 determines whether the switchable display of all the extracted image regions 600 has been finished or not (Step S380). If the controller 31 determines that the switchable display of all the image regions 600 has not been finished (Step S380: NO), the processing returns to Step S350. In this case, the controller 31 increments the variable N (N=N+1). Every time displaying of one image region 600 is finished, the controller 31 may display a button for selecting whether the display is switched to the next image region. Thus, the display can be switched to the next image region 600 by one-touch operation.

The controller 31 sequentially displays the extracted second and subsequent image regions on the interpretation screen 331. Specifically, as shown in FIG. 12C, the first image region 600a displayed on the interpretation screen 331 is switched to the second image region 600b, in response to input of a display instruction by the user. In the present embodiment, for the second lesion candidate region 60b, for example, a false positive confirmation instruction indicating a false positive is accepted. In the present embodiment, for example, a case where no operation is performed by the user is regarded as input of the false positive confirmation instruction. To indicate that the user has confirmed that the second lesion candidate region 60b is a false positive, the controller 31 makes the indication 80 hidden in the second image region 600b. When input of the interpretation determination result is finished, the user performs an operation, such as of pressing a button for transition to the next image region, for example.

As shown in FIG. 12D, the second image region 600b displayed on the interpretation screen 331 is switched to the third image region 600c, in response to input of a display instruction by the user. In the present embodiment, for example, for the third lesion candidate region 60c, e.g., a new positive confirmation instruction indicating a new positive is accepted. Upon acceptance of the positive confirmation instruction for the third lesion candidate region 60c, the controller 31 displays the indication 80 with “CHECKED” on the third image region 600c as shown in FIG. 12E, thus changing the display mode. When input of the interpretation determination result is finished, the user performs an operation, such as of pressing a button for transition to the next image region, for example.

If the controller 31 determines that the switchable display of all the extracted medical images 50 has been finished (Step S380: YES), the processing proceeds to Step S390. For example, if the switchable display of all the medical images 50 is finished, the controller 31 may display a dialog box indicating end of display on the interpretation screen 331. If a confirmation button in the dialog box is selected, the processing proceeds to the next Step S390.

On the other hand, in Step S340, if the controller 31 determines that the display instruction for displaying in a sequentially switched manner has not been accepted (Step S340: NO), the processing proceeds to Step S400, for example. For example, this is a case where the user does not use the switchable display function of the lesion candidate regions 60, and executes interpretation determination of the lesion candidate region 60 on the medical image 50 displayed on the interpretation screen 331. The controller 31 accepts the confirmation instruction by the user in accordance with the interpretation determination result, for the lesion candidate regions 60 displayed on the interpretation screen 331 (Step S400). A method similar to that in the visually listing case described above can be adopted as the input method for the interpretation determination result. If input of the interpretation determination result is finished, the processing proceeds to Step S390.

If the interpretation diagnosis of the medical image 50 is finished, the controller 31 outputs the interpretation determination result of the image region by the user to the interpretation report, and transmits data indicating the interpretation report and the interpretation determination result to the medical image management server 10 (Step S390).

Note that in the case of displaying a plurality of lesion candidate regions 60 in a sequentially switched manner, the display order may be changed based on at least one of pieces of information on the lesion accuracy and the size of a lesion of each of the lesion candidate regions 60. For example, if the lesion accuracy of the lesion is high in the order of the first lesion candidate region 60a, the third lesion candidate region 60c, and the second lesion candidate region 60b, the first lesion candidate region 60a, the third lesion candidate region 60c, and the second lesion candidate region 60b may be displayed in a switchable manner in the descending order of the lesion accuracy. Conversely, the second lesion candidate region 60b, the third lesion candidate region 60c, and the first lesion candidate region 60a may be displayed in a switchable manner in the ascending order of the lesion accuracy. The same is true for the size of the lesion as well as for the lesion accuracy of the lesion. In consideration of both the lesion accuracy and size of the lesion, the display order of the lesion candidate regions 60 may be changed.

The interpretation determination result of the user for the lesion candidate region 60 detected by the computer processing may be anything other than the indication 80 with “CHECKED”. For example, as described in the first embodiment, the display state of at least one of the concentration, the display position, and size of the image region 600 may be changed in accordance with the interpretation determination result by the user. At least one of the concentration, the display position, and the size of the image region 600, and the indication 80 with “CHECKED” described above may be combined.

As described above, according to the second embodiment, the functionalities of the apparatus/system, especially graphics techniques are improved similarly to the first embodiment. Specifically, the user can perform interpretation while visually identifying the image regions 600 including the lesion candidate regions 60 displayed in the sequentially switched manner on the interpretation screen 331. In other words, the images where only the image regions 600 including the lesion candidate regions 60 are extracted from the medical image 50 are sequentially confirmed one by one, thus allowing secondary interpretation. Accordingly, improvement in the user's interpretation working efficiency can be facilitated.

According to the second embodiment, in a case of interpretation and verification for the lesion candidate regions 60 displayed in the switchable manner, the interpretation determination result of the interpretation can be input by a left-clicking operation or the like in the image region 600. In this case, the identification function of the controller 31 automatically identifies the interpretation determination result. Accordingly, the interpretation determination result in the interpretation can be easily input and improvement in the user's interpretation working efficiency can be facilitated.

According to the second embodiment, in response to input of the interpretation determination result by the user, the indication 80 indicating that the user has confirmed the analysis result by the computer is displayed in the image region 600. Consequently, it is possible to easily identify whether the user has confirmed presence or absence of a positive in the lesion candidate region 60 or not.

### Third Embodiment

In a third embodiment, the lesion candidate region on the current medical image 50, and the lesion candidate region on the previous medical image at the same site of the same patient are compared with each other. According to the comparison result, the display mode of the lesion candidate region on the current medical image 50 is changed. Note that the components and operations identical to those of the medical image display system 100 and the like according to the first embodiment described above are assigned the identical symbols, and detailed description thereof is omitted.

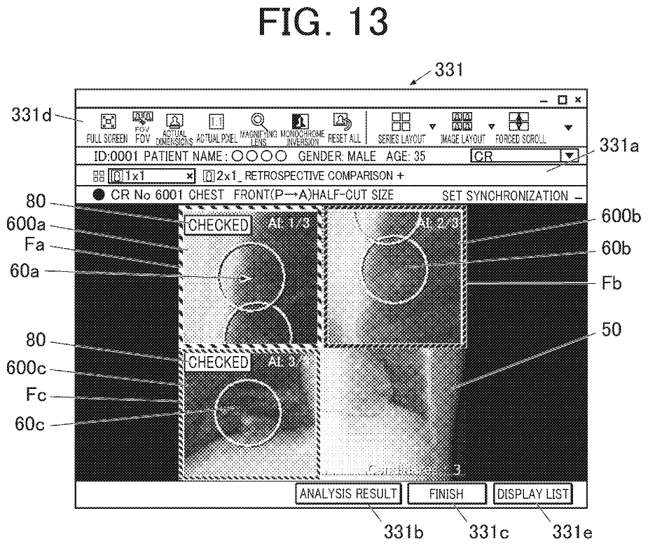

FIG. 13 shows a display example of the image regions 600 including the lesion candidate regions 60 visually listed on the interpretation screen 331 according to the third embodiment. Note that in the third embodiment, similar to the first embodiment, a case is described where three locations of the first lesion candidate region 60a, the second lesion candidate region 60b, and the third lesion candidate region 60c on the medical image 50 are detected.

In the third embodiment, if there is a previous medical image at the same site of the same patient with respect to the current medical image 50, the controller 31 obtains the interpretation determination result for the lesion candidate region 60 detected through AI analysis by the computer for the previous medical image. Note that the current image is referred to as a first medical image 50, and the previous medical image is referred to as a second medical image. The second medical image is obtained from, for example, the medical image management server 10. The controller 31 executes an identification function, which compares the lesion candidate region on the first medical image 50 with the lesion candidate region on the second medical image. The controller 31 executes a display function of changing the display mode in accordance with the comparison result.

If the first lesion candidate region 60a is determined to be positive in both the first medical image 50 and the second medical image, the possibility of a positive is high. Accordingly, the controller 31 displays the first image region 600a by surrounding the region with a purple (indicated by hatching in FIG. 13) frame Fa indicating a positive. If the second lesion candidate region 60b is determined to be positive on the first medical image 50, and the lesion candidate region 60 is not detected as a lesion candidate on the second medical image, the possibility of a positive is low. Accordingly, the controller 31 displays the second image region 600b by surrounding the region with a gray (indicated by hatching in FIG. 13) frame Fb indicating a false positive. The controller 31 displays the third lesion candidate region 60c found as a result of interpretation diagnosis by the user himself/herself by surrounding the region with a red (indicated by hatching in FIG. 13) frame Fc indicating a new positive. Note that the colors of the frames Fa, Fb, and Fc are not limited to the colors in the present embodiment. The frames may be made different by the types of patterns, such as hatching, as shown in FIG. 13.

The controller 31 accepts the confirmation instruction by the user in accordance with the interpretation determination result, for the lesion candidate regions 60 visually listed on the interpretation screen 331. Upon acceptance of the confirmation result by the user, the controller 31 changes the display mode of the image region 600 including the lesion candidate region 60. For example, similar to the first embodiment, upon acceptance of a positive confirmation result from the user, the controller 31 displays indications 80 with “CHECKED” respectively for the first lesion candidate region 60a and the third lesion candidate region 60c. Upon acceptance of the false positive confirmation result for the second lesion candidate region 60b, the controller 31 makes the indication 80 with “CHECKED” hidden.

As described above, according to the third embodiment, the functionalities of the apparatus/system, especially graphics techniques are improved similarly to the first and second embodiments. Specifically, the lesion candidate region detected by the computer on the previous medical image is compared with the lesion candidate region 60 detected by the computer on the current medical image 50. According to the comparison result, the display mode of the image region 600 is changed. Consequently, the user can perform secondary interpretation for each lesion candidate region 60, with reference to the display mode of the image region 600, i.e., the previous analysis result by the computer. Accordingly, the user can highly accurately perform secondary interpretation.

The embodiments of the present disclosure have been described in detail with reference to the accompanying drawings. However, the technical scope of the present disclosure is not limited to such examples. It is understood that techniques according to which a person of ordinary skill in the art of the present disclosure would have easily been able to make various changes and modifications in a range of the technical ideas described in the scope of claims naturally belong to the technical scope of the present disclosure.

For example, in the first embodiment, the example of visually listing the image regions 600 is described, and in the second embodiment, the example of displaying the image regions 600 in the sequentially switched manner is described. Alternatively, these techniques may be combined. Specifically, the interpretation screen 331 shown in FIG. 5 may be provided with both the image list display button 331e in the first embodiment, and the switch display button 331f in the second embodiment, thus allowing the user to select any of the buttons.

The instructions to be executed by the medical image display systems 100 according to the first to third embodiments may be stored in a computer connected to a network, such as the Internet, and downloaded via the network, thus being provided.

Although embodiments of the present invention have been described and illustrated in detail, the disclosed embodiments are made for purposes of illustration and example only and not limitation. The scope of the present invention should be interpreted by terms of the appended claims.

What is claimed is:

1. A non-transitory computer readable recording medium storing instructions causing a computer to execute:
extracting, from a medical image, an image region including a lesion candidate region obtained by processing the medical image;
causing a display to display the image region; and
identifying a confirmation result of the image region.

2. The recording medium according to claim 1, wherein
the instructions cause the computer to execute identifying an interpretation determination result of the lesion candidate region in the image region.

3. The recording medium according to claim 2, wherein
the instructions cause the computer to execute:
extracting, from the medical image, an image region including a lesion candidate region designated by a user; and
identifying an interpretation determination result of the lesion candidate region designated by the user.

4. The recording medium according to claim 1, wherein
the instructions cause the computer to execute:
displaying an indication for confirming a state of the lesion candidate region; and
associating the lesion candidate region with the indication.

5. The recording medium according to claim 4, wherein
when extracting lesion candidate regions, the instructions cause the computer to execute associating the lesion candidate regions, with indications for respectively confirming the lesion candidate regions, on one-to-one basis.

6. The recording medium according to claim 1, wherein
the image region is configured as a plurality of image regions,
the image regions respectively include lesion candidate regions different from each other, and
the instructions cause the computer to visually list the image regions.

7. The recording medium according to claim 1, wherein
the image region is configured as a plurality of image regions,
the image regions respectively include lesion candidate regions different from each other, and
the instructions cause the computer to execute:
displaying a first image region among the image regions, and
switching the displaying from the first image region to a second image region in response to an input by a user.

8. The recording medium according to claim 2, wherein
the interpretation determination result is at least one of positive, negative, false positive, false negative, and pending.

9. The recording medium according to claim 2, wherein
the instructions cause the computer to execute outputting the interpretation determination result to an interpretation report.

10. The recording medium according to claim 9, wherein
the instructions cause the computer to execute pasting an image region where at least one of positive and false negative is selected as the interpretation determination result, as a reference image, to the interpretation report.

11. The recording medium according to claim 4, wherein
the instructions cause the computer to execute displaying the image region including the lesion candidate region, and the indication that is associated with the lesion candidate region and for confirming the state of the lesion candidate region, to overlap the medical image.

12. The recording medium according to claim 11, wherein
the instructions cause the computer to extract, from the medical image, the image region that is associated with the indication and includes the lesion candidate region.

13. The recording medium according to claim 6, wherein
the instructions cause the computer to execute changing a display order, based on information on at least one of a lesion accuracy and a size of a lesion displayed in the lesion candidate region.

14. The recording medium according to claim 1, wherein
the instructions cause the computer to change a display mode of the image region, in accordance with the confirmation result of the image region.

15. The recording medium according to claim 4, wherein
the instructions cause the computer to execute changing at least one of a concentration, position, and size of the image region, and
the indication includes the at least one of the concentration, position, and size of the image region.

16. The recording medium according to claim 1, wherein
when assuming the medical image as a first medical image, the instructions cause the computer to execute obtaining a confirmation result of the lesion candidate region obtained by processing a second medical image at an identical site of an identical patient imaged before the first medical image, and comparing the confirmation result of the lesion candidate region on the first medical image with the confirmation result of the lesion candidate region on the second medical image, and
the instructions cause the computer to execute changing a display mode of the image region in accordance with a comparison result.

17. A medical image display apparatus comprising a hardware processor that:
extracts, from a medical image, an image region including a lesion candidate region obtained by processing the medical image;
causes a display to display the image region; and
identifies a confirmation result of the image region.

18. A medical image display method, causing a hardware processor to execute:
extracting, from a medical image, an image region including a lesion candidate region obtained by processing the medical image;
causing a display to display the image region; and
identifying a confirmation result of the image region.
